(19) United States
(12) Patent Application Publication
(10) Pub. No.: US 2025/0325373 A1
(43) Pub. Date: Oct. 23, 2025

(54) Method for Estimating a Value Representative of a Fluid Pressure in an Inflatable Element of an Implantable Medical Device

(71) Applicant: UROMEMS, EYBENS (FR)

(72) Inventors: Gaby BOU TAYEH, GRENOBLE (FR); Lyes DJOUMI, GRENOBLE (FR)

(21) Appl. No.: 18/869,958

(22) PCT Filed: Jun. 9, 2023
(86) PCT No.: PCT/EP2023/065495

§ 371 Date: Nov. 27, 2024

(30) Foreign Application Priority Data
Jun. 9, 2022 (FR) FR2205563

(51) Int. Cl. A61F 2/26; A61F 2/00

(52) U.S. Cl. CPC A61F 2/26; A61F 2/0004

(57) ABSTRACT

The invention relates to a method for estimating a value representative of a first pressure, the first pressure being a fluid pressure in an inflatable element (3) of an implantable medical device (10) comprising a fluid reservoir (5) having a selectively variable volume, the reservoir (5) being able to deform under the effect of a variation in atmospheric pressure, and the inflatable element (3) being in fluid communication with the reservoir (5). The method comprises the steps, implemented by a data processing and control unit (200) of the device, of: a) determining a value representative of a second pressure, the second pressure being a fluid pressure in the reservoir (5); b) estimating the value representative of the first pressure from the value representative of the second pressure and from a parameter (L) that is dependent on a value representative of a maximum variation in fluid pressure in the inflatable element (3) caused by a variation in atmospheric pressure.

### GENERAL TECHNICAL FIELD

This invention relates to a method for estimating a value representative of a fluid pressure in an inflatable element of an implantable medical device comprising a fluid reservoir of selectively variable volume.

### PRIOR ART

A number of medical devices take the form of systems which can be implanted in the body of a human or animal individual. In particular, such devices may be artificial urinary sphincters to combat urinary incontinence, gastric bands or rings suitable for restricting the stomach for the purpose of combating obesity, inflatable penile implants used for erectile prostheses, etc.

In a manner known per se, the implantable system can be hydraulic in operation and may particularly comprise a fluid reservoir of variable volume and an inflatable element containing a variable volume of fluid. The inflatable element is in fluid connection with the fluid reservoir of variable volume, so as to be able to transfer fluid from the reservoir to the inflatable element, and vice versa.

In the case of an occlusive implantable system such as an artificial urinary sphincter, the inflatable element is an inflatable occlusive cuff capable of selectively occluding an anatomical duct such as a urethra in men, or a vesical neck in women. Fluid can be transferred from the reservoir to the cuff to increase the pressure exerted on the duct, and conversely from the cuff to the reservoir to decrease the pressure exerted on the duct. Thus, according to the volume of fluid in the cuff, a more or less strong pressure can be exerted on the anatomical duct to be occluded.

Elements of the implantable fluid system can be sensitive to variations in atmospheric pressure (for example due to variations in altitude) and deform. For example, the reservoir of the implantable fluid system may comprise an elastically deformable part which deforms according to variations in atmospheric pressure. This deformation can cause an uncontrolled injection of fluid into the cuff or extraction of fluid from the cuff and therefore an increase or decrease in the fluid pressure in the cuff.

However, such variations in fluid pressure in the cuff are to be avoided. In the case of artificial urinary sphincters, overpressures are liable to damage the tissue around which the cuff is arranged, for example by causing lesions of this tissue. It is therefore necessary to control these overpressures in order to limit the corresponding risk of damage to the tissue. This involves being capable of estimating the pressure variations in the inflatable element to then be able to compensate for them where applicable.

### SUMMARY OF THE INVENTION

An aim of the invention is to reliably compute the fluid pressure in the inflatable element of an implantable medical device.

Another aim of the invention is to reliably compute the atmospheric pressure to which the implantable medical device is subjected.

This invention thus relates, according to a first aspect, to a method for estimating a value representative of a first pressure, the first pressure being a fluid pressure in an inflatable element of an implantable medical device comprising a fluid reservoir of selectively variable volume, the reservoir being able to deform under the effect of a variation in atmospheric pressure, the inflatable element in fluid connection with said reservoir comprising the steps, implemented by a data control and processing unit of the device, of:
a) determining a value representative of a second pressure, the second pressure being a fluid pressure in the reservoir,
b) estimating the value representative of the first pressure based on the value representative of the second pressure and on a parameter dependent on a value representative of a maximum variation in fluid pressure in the inflatable element caused by a variation in atmospheric pressure.

According to advantageous and non-limiting features, taken alone or in any combination whatsoever:
the value representative of the second pressure is determined based on at least one value representative of the fluid pressure in the reservoir measured by a sensor comprised in the implantable medical device;
the value representative of the first pressure is determined based on a value representative of the reference second pressure determined based on a value representative of the fluid pressure in the reservoir measured by the sensor;
the value representative of the first pressure is determined based on a value representative of a reference first pressure, the value representative of the reference first pressure being determined based on the value representative of the reference second pressure measured by the sensor and on a value representative of a reference atmospheric pressure measured by a barometer;
said barometer is disposed on an outer wall of the box 1 or in the box 1;
said barometer is comprised in an external control element suitable for exchanging data with the implantable medical device and in which the value representative of the reference atmospheric pressure is measured following a command implemented by an individual in which the device is implanted via the external control element;
the value representative of a reference first pressure is updated at each implementation of a command based on an updated value representative of a reference atmospheric pressure and on an updated value representative of a reference second pressure;
the value representative of a reference first pressure is updated at least once a day based on an updated value representative of a reference atmospheric pressure and on an updated value representative of a reference second pressure;
the value representative of a reference second pressure is updated at least once a day, preferably, at least three times a day, still preferably, at least five times a day;
the value representative of the second pressure is an average or a median of a plurality of values representative of the fluid pressure in the reservoir;
the value representative of the second pressure is determined based on an average or a median of at least three values representative of the fluid pressure in the reservoir;
the value representative of the second pressure is determined based on a value representative of a first variation in fluid pressure in the reservoir caused by a variation in the volume of the reservoir implemented by an actuator of the device;
the value representative of the second pressure is determined based on a value representative of a second variation in fluid pressure in the reservoir caused by a change in orientation of the device;
the value representative of the first pressure is computed based on the following term:

deuxième_P^{L}
deuxième_P being the value representative of the second pressure and L being said parameter dependent on the maximum variation in pressure in the inflatable element caused by the variation in atmospheric pressure;
   said parameter is obtained beforehand based on one or more measurements of a value representative of a third variation in a fluid pressure in the reservoir caused by one or more variations in atmospheric pressure;
   the value representative of the first pressure is determined by computing:

$$\frac{deuxième\_P {\_ref}^{L}}{{deuxième\_P}^{L}} \times première\_P \_ref$$

première_P_ref being a value representative of a reference first pressure and
deuxième_P_ref being a value representative of a reference second pressure.

According to a second aspect, the invention relates to a method for estimating a value representative of an atmospheric pressure to which an implantable medical device is subjected, comprising the previously described estimating method, and comprising a step c) of estimating the value representative of an atmospheric pressure by subtracting the value representative of the second pressure from the value representative of the first pressure.

According to a third aspect, the invention relates to an implantable medical device comprising a fluid reservoir of variable volume, an inflatable element in fluid connection with said reservoir and a data control and processing unit, said unit being configured to implement the previously described estimating methods.

According to advantageous and non-limiting features, taken alone or in any combination whatsoever:
the device is configured to be implanted in a human or animal body to selectively shut off an anatomical duct of said human or animal body taken from among at least one of the following ducts: a urethra, a gastric duct, a colon and a rectum;
the device comprises an inflatable element of elongated shape configured to be used as a penile implant.

According to a fourth aspect, the invention relates to an assembly comprising a previously described implantable medical device and an external control element suitable for exchanging data with the implantable medical device and suitable for being used by an individual in which the medical device is implanted, wherein the implantable medical device and the external control element comprise communication means suitable for communicating with one another.

According to a fifth aspect, the invention relates to a computer program product comprising code instructions for executing a previously described method, when said program is executed by an electronic control unit.

According to a sixth aspect, the invention relates to a storage means readable by an item of computer equipment on which a computer program product comprises code instructions for executing a previously described method.

### DESCRIPTION OF THE FIGURES

Other features and advantages of this invention will become apparent on reading the following description of a preferred embodiment. This description will be given with reference to the appended figures in which:

FIG. 1 illustrates an overall view of a medical device implantable in the body of an individual and a control element external to the body of the individual.

FIG. 2 is a schematic section view of the inside of the box according to an embodiment;

FIG. 3 shows the steps of the method for estimating a first pressure;

FIG. 4 is a graph showing the evolutions of different pressures as a function of an increase in altitude;

FIG. 5 shows the steps of the method for estimating an atmospheric pressure.

### DETAILED DESCRIPTION OF THE INVENTION

### Device

According to a first aspect, provision is made for a medical device implantable in an individual. The term “individual” should be understood to mean, in this text, a human being or an animal. The device is an active implantable medical device able to occlude a natural duct such as for example a urethra (in men), the vesical neck (in women), a gastric duct, a colon or else a rectum. In a scenario of application to a urethra or to a vesical body, the device in particular makes It possible to combat urinary incontinence by means of an artificial sphincter capable of shutting off the urethra or the vesical neck. However, the proposed device is more generally a device comprising a fluid circuit sensitive to variations in pressure, particularly generated by variations in altitude. Other forms that the device can take can in particular include penile implants and gastric constriction bands.

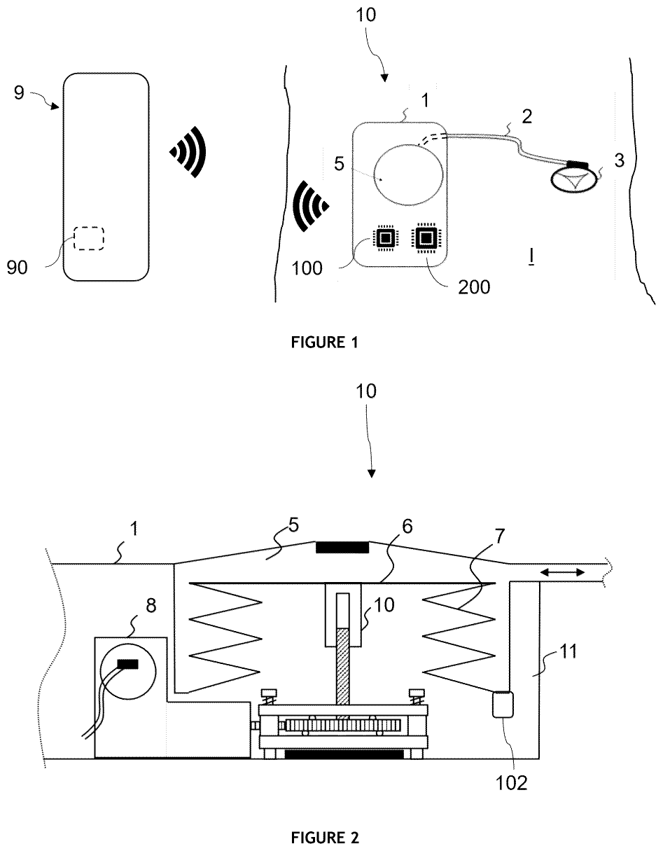

A medical device implantable in a human or animal body is illustrated by way of non-limiting example in FIGS. 1 and 2.

The implantable device 10 comprises:
a sealed box 1 containing a gas,
an inflatable element 3 suitable for being implanted in the body of the individual outside the box,
a fluid circuit comprising a fluid reservoir 5 having a variable fluid volume arranged in the box and a fluid connection 2 between said reservoir 5 and said inflatable element 3,
an actuator 8 arranged in the box 1 and mechanically coupled to a part of the fluid reservoir 5 to selectively vary the volume of fluid in said reservoir,
a control unit 100 configured to control the actuator 8 so as to transfer fluid between the reservoir 5 and the inflatable element 3,
a data control and processing unit 200 configured to implement a method for estimating a value representative of a fluid pressure in the inflatable element 3, the so-called “first pressure”.

The fluid circuit is suitable for being filled with a fluid. A variation in the volume of the reservoir 5 causes a variation in the pressure in the fluid circuit. More particularly, a decrease in the volume of the reservoir 5 causes a transfer of fluid from the reservoir 5 to the inflatable element 3, and causes an increase in the pressure in the fluid circuit. Conversely, an increase in the volume of the reservoir 5 causes a transfer of fluid from the inflatable element 3 to the reservoir 5, and causes a decrease in the pressure in the fluid circuit.

The reservoir 5 is preferably a fluid reservoir of variable volume liable to deform under the effect of a variation in atmospheric pressure. The reservoir 5 can therefore comprise an elastically deformable part which deforms as a function of variations in atmospheric pressure. This deformation can cause a variation in the volume of the reservoir 5 and therefore fluid transfers between the reservoir 5 and the inflatable element 3.

The reservoir 5 further comprises an orifice used to transfer the fluid from the reservoir 5 to the inflatable element 3 via the fluid connection 2 or from the inflatable element 3 to the reservoir 5 via the fluid connection 2.

The fluid connection 2 may consist in a tube 2 disposed between the reservoir 5 and the inflatable element 3. A first end of the tube 2 opens into the reservoir 5, and a second end of the tube 2 opens into the inflatable element 3.

The inflatable element 3 can be an inflatable occlusive cuff, in particular when the device 10 is an artificial urinary sphincter. The inflatable occlusive cuff 3 filled with fluid can be adapted to completely or partially surround the duct to be occluded.

In a variant, the inflatable element 3 can be an inflatable penile implant, and thus have an elongated shape, in particular when the device 10 is an erectile prosthesis.

The box 1, the fluid connection 2 and the inflatable element 3 are suitable for being implanted into the body of an individual I, the contours of which are schematically represented on FIG. 1 on either side of this assembly.

The box 1, in particular the inner volume 11 of the box 1 surrounding the reservoir 5, contains a gas, for example an inert gas.

The actuator 8 is suitable for controlling a variation in the volume of the reservoir 5. The actuator 8 is controlled by the control unit 100. In a certain embodiment, the actuator 8 is suitable for controlling a linear displacement of the movable wall 6, the bellows 7 being suitable for extending or compressing as a function of said linear displacement of the movable wall 6 controlled by the actuator 8.

The actuator 8 can be chosen from among any electromechanical system making it possible to convert an electrical energy into a mechanical movement with the requisite power to enable the displacement, at a required force and speed, of the movable wall 6 of the reservoir 5 of variable volume. The actuator 8 can in particular be a piezo-electric actuator, an electromagnetic actuator which may comprise a brushed or brushless electromagnetic motor coupled or not coupled to a reduction gear, an electro-active polymer or a memory alloy.

The control unit 100 is configured to control the actuator 8 so as to displace the movable wall 6 of the reservoir 5 to a position corresponding to the determined volume. More specifically, in the example illustrated in FIG. 2, the control unit 100 is suitable for sending an order to the motor of the actuator 8 to run in one direction or the other according to whether an increase or a decrease in the volume of the reservoir 5 is required.

Advantageously, the box 1 encloses a reservoir sensor 102 suitable for measuring a value representative of the fluid pressure in the reservoir 5, the so-called “second pressure”. The reservoir sensor 102 may for example be a force sensor or a pressure sensor.

Particularly advantageously, an external control element 9, such as a remote control, external to the body of the patient, is usable by the patient or a third party to communicate in a wireless manner with the medical device 10, for example by radio frequency.

In certain embodiments, a barometer 90, i.e. an atmospheric pressure sensor, is comprised in the device 10, for example in the box 1. According to another embodiment, the barometer 10 is arranged on an outer wall of the box 1 and is configured to communicate with the device 10. Still according to another embodiment, the barometer 90 can be arranged in the control element 9 external to the body of the individual in which the device 10 is implanted. The barometer 90 is suitable for measuring a value representative of an atmospheric pressure to which the implantable medical device 10 is subjected. In the case of a barometer 90 disposed in the external control element 9, a measurement of a value representative of the atmospheric pressure can thus be taken via the external control element 9, for example when the patient himself activates a command of the external control element 9.

Alternatively or in a combination, the barometer 90 can also be configured to implement a measurement of atmospheric pressure at a predetermined frequency.

### Assembly

According to a second aspect, provision is made for an assembly comprising an implantable medical device 10 as described above, and an external control element 9 suitable for being used by an individual, for example by an individual in which the system is implanted. The implantable medical device 10 and the external control element 9 comprise communication means suitable for communicating with one another in a wireless manner, for example by radio frequency. The communication means of the implantable device 10 may be integrated into the box 1.

### Methods

According to a third aspect, provision is made for a method for estimating a value representative of a first pressure, the first pressure being a fluid pressure in the inflatable element 3 of the implantable medical device 10. This value makes it possible to estimate the fluid pressure. It is particularly beneficial to estimate this pressure in order to be alerted when this pressure is too high or too low. Specifically, if the fluid pressure in the inflatable element 3 is too high, this can cause damage to the tissue of the anatomical duct that the inflatable element 3 surrounds. Conversely, if the fluid pressure in the inflatable element 3 is too low, the anatomical duct surrounded by the inflatable element 3 is liable to be insufficiently shut off which can cause, in the example scenario in which the duct is a urethra, an episode of incontinence.

Moreover, the estimation of a value representative of the first pressure makes it possible to estimate a value representative of the atmospheric pressure to which the device 10 is subjected. As explained previously, a variation in atmospheric pressure can cause deformations of the reservoir 5 of the device 10. These deformations may lead to an uncontrolled injection of fluid into the inflatable element 3 or extraction of fluid from the inflatable element 3, and therefore an increase or decrease in the fluid pressure in the inflatable element 3.

The variations in atmospheric pressure or fluid pressure in the inflatable element 3 are therefore phenomena to be monitored to avoid damage to tissue of the anatomical duct or failure to shut off the anatomical duct.

The method described hereinafter comprises the estimation of values representative of pressures. In particular, the estimated values can for example include the estimation of a value representative of a first pressure, a value representative of a second pressure, and also a value representative of an atmospheric pressure. The first pressure is a fluid pressure in the inflatable element 3 of the implantable medical device 10. The second pressure is a fluid pressure in the reservoir 5. The atmospheric pressure is the atmospheric pressure to which the device 10 and therefore the reservoir 5 is subjected. A value representative of a pressure is a value that makes it possible to estimate the pressure or which allows, if one has several values representative of pressure, the evaluation of the variations in pressure. A value representative of a pressure can be a value of absolute pressure expressed for example in Pascals. A value representative of a pressure can also be a value of force (for example a force exerted on the reservoir 5 or on the inflatable element 3). The proposed examples of values representative of pressure are not limiting and one could envision any other type of relevant values representative of pressure.

For the sake of brevity, in the remainder of the description, the concepts of the values representative of pressure will be regularly referred to simply as “pressures”. For example, the value representative of a first pressure will be referred to as “first pressure” but it will be understood that this expression “first pressure” denotes the value representative of first pressure.

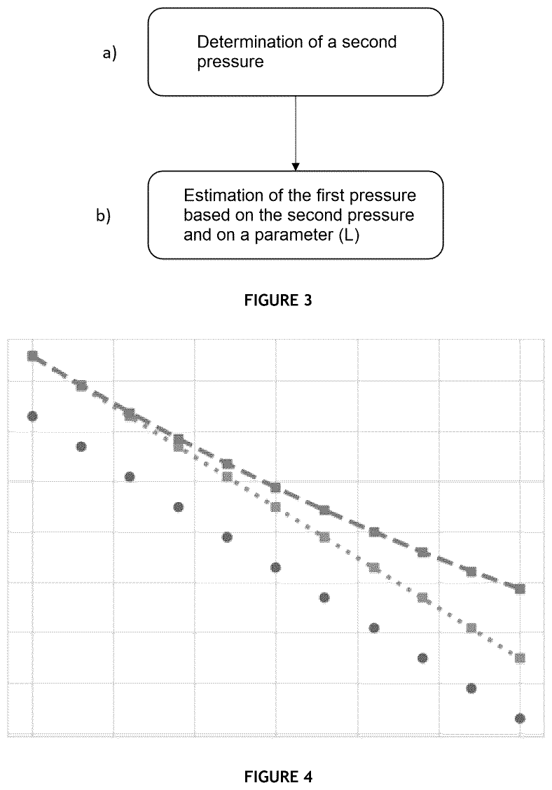

With reference to FIG. 3, the method for estimating the first pressure, i.e. the fluid pressure in the inflatable element 3, comprises a step a), implemented by the data control and processing unit 200, of determining a second pressure, i.e. the fluid pressure in the reservoir 5. The second pressure will be written deuxième_P in the remainder of the text.

According to an embodiment, the second pressure is determined based on at least one value representative of the fluid pressure in the reservoir 5 measured by the reservoir sensor 102.

Advantageously, the second pressure is an average or a median of a plurality of values representative of the fluid pressure in the reservoir 5. In other words, a plurality of values representative of the fluid pressure in the reservoir 5 are acquired by the reservoir sensor 102 over a given time interval and an average or a median is computed based on the plurality of values in order to obtain the second pressure. Still preferably, the second pressure is determined based on an average or a median of at least three values representative of the fluid pressure in the reservoir 5. The median or the average of the plurality of values representative of the fluid pressure in the reservoir 5 is written M_deuxièmes_P and, in a certain embodiment, deuxième_P is equivalent to M_deuxièmes_P. In this case, the fact of computing an average or a median of several values representative of the fluid pressure in the reservoir 5 measured in succession over time makes it possible to evaluate the second pressure over time as a function of variations in atmospheric pressure. The resulting second pressure value is a smoothed value which takes into account the evolution of the fluid pressure in the reservoir 5 as a function of variations in atmospheric pressure. Preferably, the values representative of the fluid pressure in the reservoir 5 used to compute the median or the average are measured at intervals of between 10 seconds and 8 minutes, preferably approximately equal to 2 minutes.

So as to estimate the second pressure in the most realistic and accurate manner possible, different terms are advantageously computed which are taken into account in the computation of the second pressure.

In this regard, according to a preferred embodiment, the second pressure is determined based on a value representative of a first variation in fluid pressure in the reservoir 5 Δₚᵣₑₛₛᵢₒₙ caused by a variation in the volume of the reservoir 5 implemented by an actuator 8 of the device 10. More accurately, when an increase or a decrease in the volume of the reservoir 5 controlled by the actuator 8 of the device 10 is implemented, a variation in fluid pressure in the reservoir 5 Δₚᵣₑₛₛᵢₒₙ takes place. The variation Δₚᵣₑₛₛᵢₒₙ is estimated by measurement of the fluid pressure in the reservoir 5 before and after the first variation in volume of the reservoir 5 controlled by the actuator 8. In this embodiment, the second pressure deuxième P is equivalent to M_deuxièmes_P+Δₚᵣₑₛₛᵢₒₙ i.e. the sum of the median or the average of the plurality of values representative of the fluid pressure in the reservoir 5 and of the value representative of the first variation in fluid pressure in the reservoir 5. Thus, according to this embodiment, the estimation of the second pressure deuxième P takes into account the variation in pressure that has been caused by the last variation in volume of the reservoir 5 controlled by the actuator 8. The estimation of the second pressure is thus an adapted/corrected estimation.

According to another preferred embodiment, the second pressure is determined based on a value representative of a second variation in fluid pressure in the tank 5 Δ_{Orientation} caused by a change in orientation of the device 10. Typically, a change in orientation of the device 10 may be caused by a change of position or posture of the individual in which the device 10 is implanted. In the case of a human for example, a change in orientation of the device 10 may be caused by the change in the position of the human from standing to lying down or conversely. A change in orientation of the device 10 is liable to cause a variation in fluid pressure in the reservoir 5. For example, when a human lies down, the fluid pressure in the reservoir 5 will increase. Δ_{Orientation} can for example be a value which changes when a change in orientation of the device 10 is detected. For example, such a change in orientation of the device can be automatically detected by means of sensors, as described in the document WO2021255388. In other scenarios, the individual can himself activate a “lying-down” mode by pressing a suitable button of its external control element 9 and Δ_{Orientation} will take a different value from the previous one, before the individual activated “lying-down” mode. The data control and processing unit 200 will then take into account Δ_{Orientation}. In this embodiment, the second pressure deuxième_P is equivalent to M_deuxièmes_P+Δ_{Orientation} i.e. the sum of the median or the average of the plurality of values representative of the fluid pressure in the reservoir 5 and of the value representative of the second variation in fluid pressure in the tank 5 caused by a change in orientation of the device 10.

If one considers the two preceding embodiments described, deuxième_P is expressed M_deuxièmes_P+Δₚᵣₑₛₛᵢₒₙ+Δ_{Orientation}. It will be understood that other terms may be taken into account to estimate the second pressure as closely to reality as possible and that the terms taken into account are not limited to those described.

With reference to FIG. 3, the method for estimating the first pressure then comprises a step b) of estimating the first pressure based on the second pressure and on a parameter L dependent on a value representative of a maximum variation in fluid pressure in the inflatable element 3 caused by a variation in atmospheric pressure. In fact, preferably, the first pressure is computed based on the term deuxième_P^{L}.

By way of illustration, the parameter L is representative of the fluid overpressure in the inflatable element 3 due to a deformation of the reservoir 5 caused by a variation in atmospheric pressure. In the event of a variation in atmospheric pressure, the reservoir 5 is liable to deform due to the elastic properties of certain of its parts. This leads to a variation in the fluid pressure in the inflatable element 3. FIG. 4 graphically illustrates the phenomenon that occurs when the atmospheric pressure to which the fluid of the reservoir 5 is subjected decreases. The plain dotted curve (first curve from the bottom of the graph) plots the evolution of the atmospheric pressure as a function of altitude. The more the altitude increases, the more the atmospheric pressure decreases. The dotted curve with squares (second curve from the bottom of the graph) plots the evolution of the second pressure, namely the fluid pressure in the reservoir 5, as a function of altitude, for a reservoir 5 that does not deform despite variations in atmospheric pressure. This second curve illustrates an ideal scenario which does not reflect reality. In this case, the second pressure evolves in a linear manner and has the same guide coefficient as the first curve illustrating the evolution of the atmospheric pressure. The more the altitude increases, and therefore the atmospheric pressure decreases, the more the second pressure decreases in a linear manner. The dashed curve with squares (third curve from the bottom of the graph) plots the evolution of the second pressure, namely the fluid pressure in the reservoir 5, as a function of altitude, for a reservoir 5 which deforms in the event of variations in atmospheric pressure. This curve illustrates what really happens. It can be seen that this curve is non-linear and the fluid pressure in the reservoir 5 does not decrease as much as in the case of a reservoir 5 which does not deform because of variations in atmospheric pressure. This non-linearity of the curve is due to the fact that, when the atmospheric pressure decreases (i.e. the altitude increases), the reservoir 5 deforms so that its volume decreases, which effects an increase in fluid pressure in the reservoir 5. The fluid pressure in the reservoir 5 therefore decreases less as a function of the decrease in atmospheric pressure than if the reservoir 5 was not deformable as illustrated in the third curve. At the same time, the decrease in the volume of the reservoir 5 causes an injection of fluid from the reservoir 5 into the inflatable element 3 which effects an increase in fluid pressure in the inflatable element 3. Thus, the non-linearity of the third curve is representative of an overpressure of the fluid in the inflatable element 3 and the greater the space between the second and third curves, the higher the overpressure of the fluid in the inflatable element 3. The parameter L makes it possible to mathematically represent this non-linearity.

For example, in the embodiment illustrated on FIG. 2, the bellows 7 forms a part of the wall of the reservoir of variable volume. The bellow is formed by a plurality of convolutions, which can be elastically deformable. The bellows 7 can be in the shape of a concertina bellows, the convolutions corresponding to folds of the concertina. In the static state, i.e. when no stress is exerted by the actuator 8 to vary the volume of the reservoir, a variation in atmospheric pressure can give rise to a deformation of the convolutions, thus varying the volume of the reservoir.

It will be understood that the preceding paragraph could be reciprocally applied to a scenario of decreasing altitude and therefore of increasing atmospheric pressure. In this case, during the increase in atmospheric pressure, the reservoir 5 deforms so that its volume increases, which effects a decrease in fluid pressure in the reservoir 5. The fluid pressure in the reservoir 5 therefore increases less as a function of the decrease in atmospheric pressure than if the reservoir 5 was not deformable. In parallel, the increase in the volume of the reservoir 5 draws fluid from the inflatable element 3 into the reservoir 5 which effects a decrease in fluid pressure in the inflatable element 3.

The parameter L is a predetermined parameter and depends on various factors. The term “predetermined” should be understood to mean that the parameter is configured following the implantation of the device 10 in an individual, which is then not intended to be regularly modified even if it is modifiable.

First of all, the parameter L depends on a value representative of a maximum variation in fluid pressure in the inflatable element 3 following a deformation of the reservoir 5 caused by a variation in atmospheric pressure. In practice, this maximum variation will be determined by empirical observation when the device 10 is implanted in an individual. More precisely, to simulate a decrease in atmospheric pressure, fluid will be injected from the reservoir 5 into the inflatable element 3. It may for example be considered that the maximum decrease in atmospheric pressure will be the maximum variation in atmospheric pressure that will be equivalent to an increase in altitude from 0 meters to 3000 meters above sea level. The volume of fluid which is intended to be injected into the inflatable element 3 from the reservoir 5 following a deformation of reservoir 5 caused by this decrease in atmospheric pressure is known and is injected into the inflatable element 3. An estimate of the fluid pressure in the inflatable element 3 is then made. In this way an estimate is made of the maximum variation in fluid pressure in the inflatable element 3 following a deformation of the reservoir 5 caused by a variation in atmospheric pressure. It should be noted that the parameter L can be obtained from a plurality of measurements of a value representative of a third variation in a fluid pressure in the reservoir 5 caused by one or more variations in atmospheric pressure. In other words, the computing procedure described in this paragraph can be implemented for different simulations of variation in atmospheric pressure.

The parameter L preferably also depends on a rate of deformation of the reservoir 5. This rate is a quantity representative of the deformability (or else of the elasticity) of the reservoir 5. This rate depends among other things on the component material or materials of the reservoir 5 and on the size of the reservoir 5. In principle, two identical reservoirs 5 having the same materials of the same size must have an identical deformation rate.

The parameter L can also depend on the size of the inflatable element 3.

As explained previously, the first pressure depends on the second pressure along with the parameter L. More precisely, the first pressure depends on the term deuxième_P^{L}.

Advantageously, the first pressure is estimated based on other quantities.

Specifically, the first pressure is preferably determined based on a reference second pressure deuxième_P_ref determined based on a fluid pressure in the reservoir 5 measured by the reservoir sensor 102. The reference second pressure initially corresponds to a measurement taken by the reservoir sensor 102 before the activation of the device 10. The reference second pressure corresponds to a reference value of the fluid pressure in the reservoir 5. The reference second pressure is advantageously updated periodically based on measurements of the reservoir sensor 102. Preferably, the reference second pressure is updated at least once a day, preferably, at least three times a day, still preferably, at least five times a day. It will here be understood that the reference second pressure is updated less frequently than the median or the average of the plurality of values representative of the fluid pressure in the reservoir 5 M_deuxièmes_P. In fact, for illustrative purposes, the second reference pressure is measured at a time to while the values representative of the fluid pressure in the reservoir 5 measured for the computation of the median or the average will be computed at different times to +X minutes (or X seconds, X hours . . . ). Then, if the median or the average is computed based on four values representative of the fluid pressure in the reservoir 5, when eight values representative of the fluid pressure in the reservoir 5 are measured, the average or the median which had been computed based on the four first values is updated and replaced by the average or the median which is computed based on the four most recent values. Moreover, from the time at which a reference second pressure is measured (and therefore the reference second pressure is updated), four new measurements of values representative of the fluid pressure in the reservoir 5 will be measured to compute the average or the median and thus update the preceding average or median value.

Furthermore, preferably, the first pressure is determined based on a reference first pressure. The reference first pressure première_P_ref is determined based on the reference second pressure and on a reference atmospheric pressure P_atm_ref measured by the barometer 90. Atmospheric pressure, first pressure and second pressure are considered as being mathematically related. Specifically, it is accepted that première pression=deuxième pression−pression atmosphérique. Consequently, it will be understood that the reference first pressure can be estimated as a function of the reference second pressure and of a reference atmospheric pressure. The reference atmospheric pressure is preferably automatically, periodically measured by the barometer 90 of the external control element 9 and/or when the individual requests it via the external control element 9. Based on the second reference pressure and on the reference atmospheric pressure, the reference first pressure is computed as follows première_P_ref=deuxième_P_ref−P_atm_ref. Advantageously, the reference first pressure is updated at least once a day. This logically implies that the reference second pressure and the reference atmospheric pressure are updated at least once a day. Alternatively, the reference first pressure is not measured and corresponds to a predetermined value.

Finally, advantageously, the first pressure is computed by the control and processing unit based on the following formula:

$$\frac{deuxième\_P {\_ref}^{L}}{{deuxième\_P}^{L}} \times première\_P \_ref .$$

Preferably, deuxième_P=M_deuxièmes_P+Δₚᵣₑₛₛᵢₒₙ+Δ_{Orientation}.

Thus, the first pressure, i.e. the fluid pressure in the inflatable element 3, is estimated by the control and processing unit. This makes it possible for example to compare this estimate to one or more thresholds of first pressure in order to determine if the first pressure is less than or greater than a certain threshold, which would be synonymous with danger or discomfiture for the individual in which the device 10 is implanted.

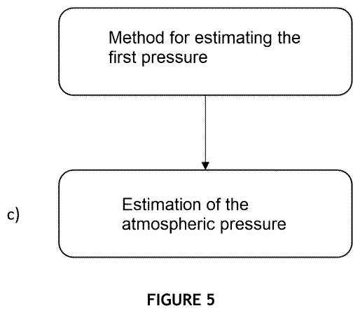

According to a fourth aspect, provision is made for a method for estimating an atmospheric pressure to which the implantable medical device 10 is subjected. With reference to FIG. 5, this second method first comprises the method for estimating the first pressure described above.

The second method further comprises a step c) of estimating the atmospheric pressure by subtracting the first pressure from the second pressure. Given that the second pressure and the first pressure have been obtained, it is enough to compute the following subtraction deuxième_P−première_P to obtain the atmospheric pressure to which the device 10 is subjected. As explained, the variations in atmospheric pressure may cause a deformation of the reservoir 5 and thus cause variations in the first pressure, i.e. in the fluid pressure in the inflatable element 3. The fact of being able to compute the atmospheric pressure makes it possible to be capable of detecting a variation in atmospheric pressure. Procedures for compensating for this variation in atmospheric pressure can then be implemented.

### Program Product and Storage Means

According to a fifth aspect, provision is made for a computer program product comprising code instructions for executing the method for estimating a value representative of the first pressure and the method for estimating a value representative of the atmospheric pressure, when said program is executed by an electronic control unit.

According to a sixth aspect, provision is made for a storage means readable by an item of computer equipment on which a computer program product comprises code instructions for executing the method for estimating a value representative of the first pressure and the method for estimating a value representative of the atmospheric pressure.

The invention is not limited to the embodiment described and shown in the appended figures. Modifications remain possible, particularly from the point of view of the composition of the various technical features or by the substitution of technical equivalents, without however departing from the general teachings.

1. A method for estimating a value representative of a first pressure, the first pressure being a fluid pressure in an inflatable element of an implantable medical device comprising a reservoir of fluid of selectively variable volume, the reservoir being able to deform under an effect of a variation in atmospheric pressure, the inflatable element in fluid connection with the reservoir comprising the steps, implemented by a data control and processing unit of the device, of:
a) determining a value representative of a second pressure, the second pressure being a fluid pressure in the reservoir,
b) estimating the value representative of the first pressure based on the value representative of the second pressure and on a parameter dependent on a value representative of a maximum variation in fluid pressure in the inflatable element caused by a variation in atmospheric pressure.

2. The estimating method as claimed in claim 1, wherein the value representative of the second pressure is determined based on at least one value representative of the fluid pressure in the reservoir measured by a sensor comprised in the implantable medical device.

3. The estimating method as claimed in claim 2, wherein the value representative of the first pressure is determined based on a value representative of the reference second pressure determined based on a value representative of the fluid pressure in the reservoir measured by the sensor.

4. The estimating method as claimed in claim 3, wherein the value representative of the first pressure is determined based on a value representative of a reference first pressure, the value representative of the reference first pressure being determined based on the value representative of the reference second pressure measured by the sensor and on a value representative of a reference atmospheric pressure measured by a barometer.

5. The estimating method as claimed in claim 4, wherein the barometer is disposed on an outer wall of the box or in the box.

6. The estimating method as claimed in claim 4, wherein the barometer is comprised in an external control element suitable for exchanging data with the implantable medical device and in which the value representative of the reference atmospheric pressure is measured following a command implemented by an individual in which the implantable medical device is implanted via the external control element.

7. The estimating method as claimed in claim 4, wherein the value representative of the reference first pressure is updated at each implementation of a command based on an updated value representative of the reference atmospheric pressure and on an updated value representative of the reference second pressure.

8. The estimating method as claimed in claim 4, wherein the value representative of the reference first pressure is updated at least once a day based on an updated value representative of the reference atmospheric pressure and on an updated value representative of the reference second pressure.

9. The estimating method as claimed in claim 3, wherein the value representative of the reference second pressure is updated at least once a day.

10. The estimating method as claimed in claim 1, wherein the value representative of the second pressure is an average or a median of a plurality of values representative of the fluid pressure in the reservoir.

11. The estimating method as claimed in claim 10, wherein the value representative of the second pressure is determined based on an average or a median of at least three values representative of the fluid pressure in the reservoir.

12. The estimating method as claimed in claim 1, wherein the value representative of the second pressure is determined based on a value representative of a first variation in fluid pressure in the reservoir caused by a variation in the volume of the reservoir implemented by an actuator of the implantable medical device.

13. The estimating method as claimed in claim 1, wherein the value representative of the second pressure is determined based on a value representative of a second variation in fluid pressure in the reservoir caused by a change in orientation of the implantable medical device.

14. The estimating method as claimed in claim 1, wherein the value representative of the first pressure is computed based on the following term:
deuxième_P^{L} [Math. 1]
deuxième_P being the value representative of the second pressure and L being the parameter dependent on the maximum variation in pressure in the inflatable element caused by the variation in atmospheric pressure.

15. The estimating method as claimed in claim 1, wherein the parameter is obtained beforehand based on one or more measurements of a value representative of a third variation in a fluid pressure in the reservoir caused by one or more variations in atmospheric pressure.

16. The estimating method as claimed in claim 1, wherein the value representative of the first pressure is determined by computing:
$$\frac{deuxième\_P {\_ref}^{L}}{{deuxième\_P}^{L}} \times première\_P \_ref \qquad [ Math . 2 ]$$
première_P_ref being a value representative of a reference first pressure and deuxième_P_ref being a value representative of a reference second pressure.

17. The estimating method as claimed in claim 1, further comprising a step c) of estimating a value representative of an atmospheric pressure to which an implantable medical device is subjected, by subtracting the value representative of the second pressure from the value representative of the first pressure.

18. An implantable medical device comprising a fluid reservoir of variable volume, an inflatable element in fluid connection with the reservoir and a data control and processing unit, the data control and processing unit being configured to implement a method as claimed in claim 1.

19. The implantable medical device as claimed in claim 18, configured to be implanted in a human or animal body to selectively shut off an anatomical duct of the human or animal body taken from among at least one of the following ducts: a urethra, a gastric duct, a colon and a rectum.

20. The implantable medical device as claimed in claim 19, comprising an inflatable element of elongated shape configured to be used as a penile implant.

21. An assembly comprising the implantable medical device as claimed in claim 18 and an external control element suitable for exchanging data with the implantable medical device and suitable for being used by an individual in which the medical device is implanted, wherein the implantable medical device and the external control element comprise communication means suitable for communicating with one another.

22. A computer program product comprising code instructions for executing a method as claimed in claim 1, when the computer program product is executed by an electronic control unit.

23. A storage means readable by an item of computer equipment on which a computer program product comprises code instructions for executing a method as claimed in claim 1.
